(19) United States
(12) Patent Application Publication
(10) Pub. No.: US 2018/0357307 A1
(43) Pub. Date: Dec. 13, 2018

(54) APPARATUS, SYSTEM AND METHOD FOR DISPLAYING A SEMANTICALLY CATEGORIZED TIMELINE

(71) Applicant: KONINKLIJKE PHILIPS N.V., EINDHOVEN (NL)

(72) Inventors: Merlijn Sevenster, Haarlem (NL); Zarko Aleksovski, Eindhoven (NL); Yuechen Qian, Lexington (MA); Thusitha Dananjaya De Silva Mabotuwana, Bothell, WA (US)

(73) Assignee: KONINKLIJKE PHILIPS N.V., EINDHOVEN (NL)

(21) Appl. No.: 15/576,040

(22) PCT Filed: May 20, 2016
(86) PCT No.: PCT/IB2016/052963

§ 371 Date: Nov. 21, 2017

Related U.S. Application Data
(60) Provisional application No. 62/174,590, filed on Jun. 12, 2015.

(51) Int. Cl. G06F 17/30; G16H 15/00; G06F 17/27; G16H 50/30; G16H 50/70

(52) U.S. Cl. CPC G06F 17/30713; G16H 15/00; G06F 17/3071; G06F 17/30707; G16H 50/70; G06F 17/2705; G06F 17/2785; G06F 17/30616; G16H 50/30; G06F 17/30734

(57) ABSTRACT

A system and method perform the steps of retrieving a report for an imaging exam; parsing out text from the report; mapping the parsed text to an ontology; automatically deriving a categorization scheme from ontology concepts extracted from the report for the imaging exam; assigning a semantic category to the imaging exam using the ontology concepts and the categorization scheme; and grouping the imaging exam with other imaging exams based on the assigned semantic category.

### BACKGROUND

Prior to conducting a radiology exam, a radiologist may examine one or more relevant prior imaging exams to establish proper context for the current study. A comprehensive radiological interpretation includes comparison against relevant prior exams. Establishing context is a non-trivial task, particularly since patient histories may include related findings across multiple clinical episodes. Existing radiology equipment may provide a patient's past imaging exams along a basic timeline. However, the timeline may be crowded with multiple exams, which increases the difficulty of establishing proper context.

Radiologists typically must familiarize themselves with a large number of prior exams in order to diagnose and treat patients in an effective manner. The use of prior studies may establish proper context for a current study. In particular, patients may frequently undergo imaging exams, resulting in a large number of prior exams to be reviewed by a radiologist. The designation “radiologist” is used throughout this description to refer to the individual who is reviewing a patient's medical records, but it will be apparent to those of skill in the art that the individual may alternatively be any other appropriate user, such as a doctor, nurse, or other medical professional.

Relevance is a context-dependent notion that is determined by a specific clinical question. There is no straightforward manual or automated method for identifying relevant prior exams. In particular, easy-to-check criteria, including modality and anatomy are not always sufficient to retrieve relevant exams to address complex clinical questions. For instance, to address complex clinical questions, a radiologist may need to know whether the patient has had a history of oncology or surgery, and may need imaging exams that reflect this history. Thus, the radiologist needs an efficient method for filtering and grouping prior imaging exams by semantic categories, to enable the radiologist to easily browse extensive histories of imaging exams and detect relevant exams on a timeline of imaging exams.

### SUMMARY

A method, comprising: retrieving a report for an imaging exam; parsing out text from the report; mapping the parsed text to an ontology; automatically deriving a categorization scheme from ontology concepts extracted from the report for the imaging exam; assigning a semantic category to the imaging exam using the ontology concepts and the categorization scheme; and grouping the imaging exam with other imaging exams based on the assigned semantic category.

A system, comprising: a non-transitory computer readable storage medium storing an executable program; and a processor executing the executable program to cause the processor to: retrieve a report for an imaging exam, parse out text from the report; map the parsed text to an ontology; automatically derive a categorization scheme from ontology concepts extracted from the report for the imaging exam; assign a semantic category to the imaging exam using the ontology concepts and the categorization scheme; and group the imaging exams with other imaging exams based on the assigned semantic category.

A non-transitory computer-readable storage medium including a set of instructions executable by a processor, the set of instructions, when executed by the processor, causing the processor to perform operations, comprising: retrieving a report for an imaging exam; parsing out text from the report; mapping the parsed text to an external ontology; automatically deriving a categorization scheme from ontology concepts extracted from the report for the imaging exam; assigning a semantic category to the imaging exam using the ontology concepts and the categorization scheme; and grouping the imaging exam with other imaging exams based on the assigned semantic category.

### BRIEF DESCRIPTION OF THE DRAWINGS

FIG. 1 shows a schematic drawing of a system according to an exemplary embodiment.

FIG. 2 shows a flow diagram of a method according to a first exemplary embodiment.

FIG. 3 shows a flow diagram of an exemplary method of step 217 for creating concept groups in FIG. 2.

FIG. 4 shows a timeline display according to a first exemplary embodiment.

FIG. 5 shows a timeline display according to a second exemplary embodiment.

### DETAILED DESCRIPTION

The exemplary embodiments may be further understood with reference to the following description and the appended drawings, wherein like elements are referred to with the same reference numerals. The exemplary embodiments relate to systems and methods for grouping imaging exams by semantic categories on a patient imaging timeline for a patient with multiple imaging exams. Although exemplary embodiments specifically describe grouping imaging exams, it will be understood by those of skill in the art that the systems and methods of the present disclosure may be used to group any type of study or exam within any of a variety of hospital settings.

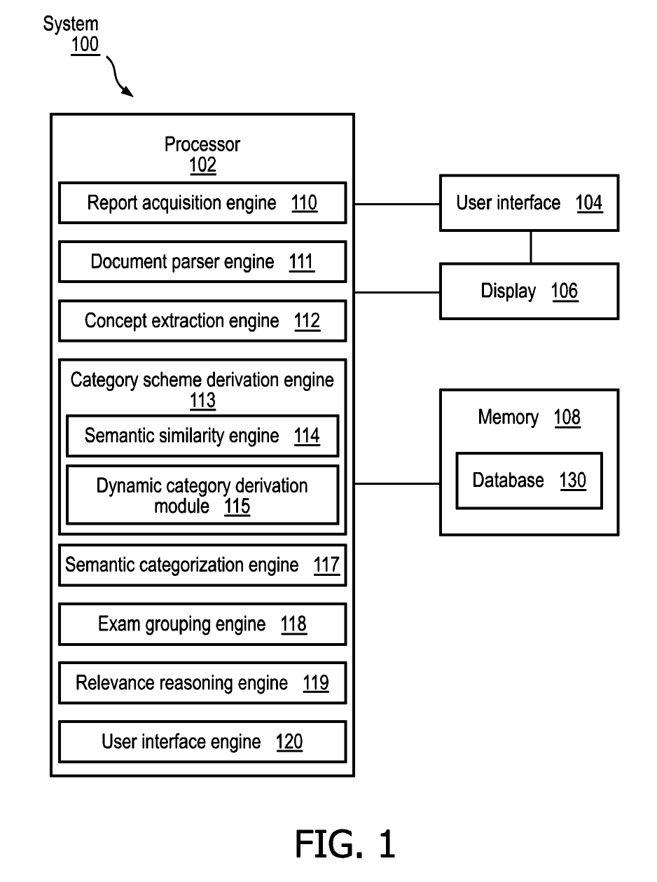

As shown in FIG. 1, a system 100, according to an exemplary embodiment of the present disclosure, groups imaging exams by semantic category. FIG. 1 shows an exemplary system 100 for filtering imaging exams by semantic categories, on a patient imaging timeline for a patient with multiple imaging exams. The system 100 comprises a processor 102, a user interface 104, and a memory 108. The memory 108 includes a database 130, which stores prior and current imaging exams, and radiology reports for a patient. Imaging exams may include exams performed on MRI, CT, CR, ultrasound, etc. Those of skill in the art will understand that the method of the present disclosure may be used to group and filter any type of imaging exam. In addition, a radiology report, for example, is a reading of results of an imaging exam for the patient and may include relevant information regarding findings and diagnoses in the image along with follow-up suggestions and recommendations. The imaging exams on a patient timeline may be viewed in, for example, a display 106 for a Picture Archiving and Communications System (PACS), and the imaging exams may be filtered and reviewed via a user interface 104.

The processor 102 includes a report acquisition engine 110, a document parser engine 111, a concept extraction engine 112, a category scheme derivation engine 113, a semantic categorization engine 117, an exam grouping engine 118, a relevance reasoning engine 119, and a user interface (UI) engine 120.

Those skilled in the art will understand that the engines 111-120 may be implemented by the processor 102 as, for example, lines of code that are executed by the processor 102, as firmware executed by the processor 102, as a function of the processor 102 being an application specific integrated circuit (ASIC), etc. The report acquisition engine 110 retrieves the report for a given imaging exam, for example, from the database 130. The document parser engine 111 parses text included in the imaging exam. For example, the document parser engine 111 may parse out headers of sections, paragraphs, and sentences in the medical narrative of the report, and may normalize the headers with respect to a pre-determined set of headers. The concept extraction engine 112 detects phrases and maps the phrases to an external ontology. Exemplary external ontologies may include SNOMED, UMLS or RadLex.

The category scheme derivation engine 113 then automatically derives a category scheme from the concepts extracted from the report of the imaging exam. In one exemplary embodiment, the category scheme is static, which means that the imaging exams are categorized according to predefined schemes that are not presently created on the basis of the reports for the imaging exams. Exemplary predefined schemes include oncology, auto-immune disorders, or cardiac disorders, etc.

In another exemplary embodiment, the category scheme is derived dynamically, which applies a method for determining the semantic similarity between two concepts. The category scheme derivation engine 113 may be implemented with several engines and modules including, for example, the semantic similarity engine 114 and the dynamic category derivation module 115. The semantic similarity may be determined based on ontology relationships between concepts, including for example, the “is-a” parent-child relationship between the concepts, e.g. a “left kidney” is-a type of “kidney.” In one exemplary embodiment, in response to two concepts from the same ontology, the semantic similarity engine 114 provides a Boolean response (yes or no) or a numerical value indicating the semantic similarity of the concepts. In another exemplary embodiment, in response to one concept, the semantic similarity engine 114 returns all semantically similar concepts.

In another exemplary embodiment, the dynamic category derivation module 115 creates groups of similar concepts, based on weights assigned to the concepts. In another exemplary embodiment, the dynamic category derivation module 115 creates groups of similar concepts, based on weights assigned to the groups. Groups with high weights may be specialized, e.g. broken down into low-weight subgroups. Or, groups with low weights may be generalized, e.g. merged with other groups with low weights. The specialization and generalization approaches create groups of concepts, where each concept group is a single category scheme. Each group may have one or more representative concepts, for example, the most general concept of the group, e.g. “respiratory disease.”

The semantic categorization engine 117 then assigns one or more semantic categories to an imaging exam from the category scheme derived by the category scheme derivation engine 113. In an exemplary embodiment, the semantic categorization engine 117 matches a given concept against the semantic category's list of ontology concepts. In another exemplary embodiment, a semantic categorization subengine attempts to establish a semantic relationship between a given input concept and the list of representative concepts through the ontology's relationships. Special traversal logic rules may be applied to restrain the iterative traversal of concepts, and if the ontology may be traversed from the input concept to a representative ontology concept for a category, the input concept belongs in the category. In another exemplary embodiment, multiple input concepts are categorized together, as a whole. For example, each input concept may be categorized, and the input concepts may be first aggregated together based on specified rules, and the aggregated input concepts are placed into a category.

The exam grouping engine 118 next groups the current imaging exam with other imaging exams into the same semantic category, based on the output of the semantic categorization engine 117. In one exemplary embodiment, if two imaging exams have been associated with the same category through the concepts extracted from the imaging exams by the semantic categorization engine 117, the exam grouping engine 118 groups imaging exams into the same semantic category. The exam grouping engine 118 also groups prior stored imaging exams into semantic categories, based on the output of the semantic categorization engine 117, according to the exemplary embodiments described above with reference to grouping the current imaging exam.

The relevance reasoning engine 119 determines whether prior imaging exams are relevant, given a current selected imaging exam. In an exemplary embodiment, the relevance reasoning engine 119 determines that all imaging exams grouped into the same semantic category by exam grouping engine 118 are relevant. The user interface engine 120 displays the timeline of imaging exams, semantic groups, and relevant imaging exams on the display 106, and aids user navigation of prior relevant and other imaging exams on the timeline via user interface 104, which may include input devices such as, for example, a keyboard, a mouse, or touch display on the display 106.

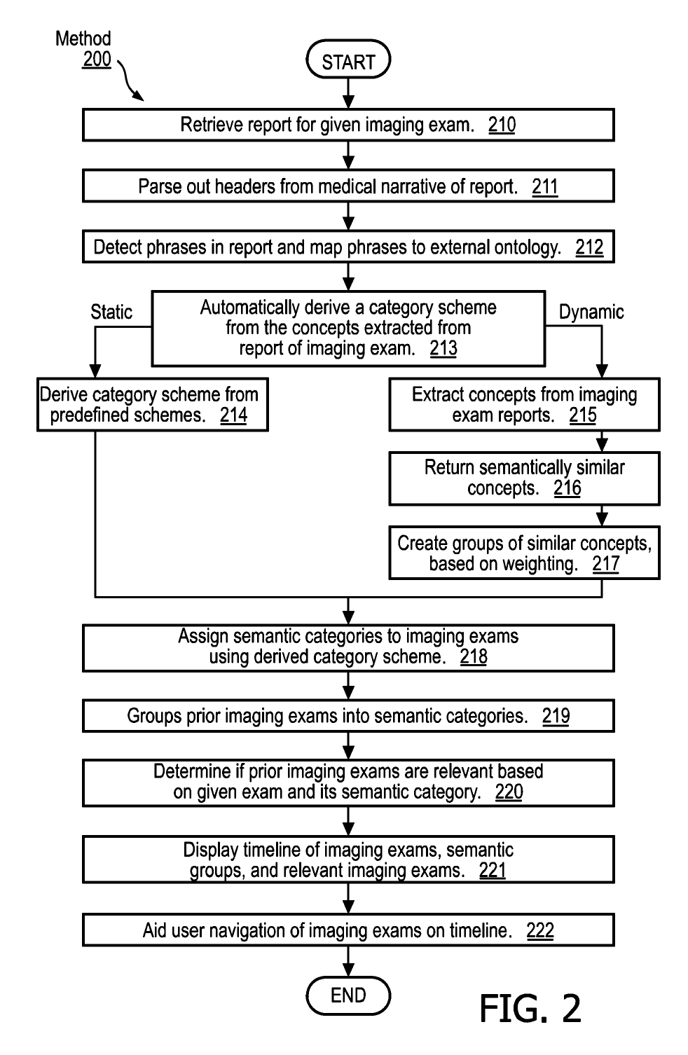

FIG. 2 shows a method 200 for filtering and grouping imaging exams by semantic categories, on a patient imaging timeline for a patient with multiple imaging exams, using the system 100 above. The method 200 comprises steps for reviewing reports for a given imaging exam, and filtering and grouping imaging exams by semantic categories, on a patient imaging exam timeline, which may be viewed on, for example, a Picture Archiving and Communications System (PACS) client.

In step 210, the report acquisition engine 110 retrieves reports for a given imaging exam. In step 211, the document parser engine 111 parses out headers of sections, paragraphs, and sentences from the medical narrative of the report. In an exemplary embodiment, the headers may then be normalized with respect to a pre-determined set of headers. For example, a pre-determined section header may be “Impression,” while a pre-determined paragraph header may be “Liver.” Rule-based or machine learning techniques may be used to implement the document parser engine 111. A maximum entropy model may be used to implement the document parser engine 111.

In step 212, the concept extraction engine 112 detects phrases in the medical narrative of the report, and maps the phrases to an external ontology, for example, SNOMED, UMLS, or Radlex. MetaMap is an exemplary concept extraction engine. It will be understood by those of skill in the art that other ontologies and concept extraction engines may be used.

In step 213, the category scheme derivation engine 113 automatically derives a category scheme from the concepts extracted from the report for the imaging exam. The category scheme is a set of categories that are used to categorize the imaging exams. Each category may correspond to a unique concept from an ontology. For example, the oncology category may correspond to the concept “cancer.” In one exemplary approach, as depicted in step 214, the category scheme is static, which means that the imaging exams are categorized according to predefined schemes that are not presently created on the basis of the reports for the imaging exams. Exemplary predefined schemes may include oncology, auto-immune disorders, cardiac disorders, infectious disorders, metabolic disorders, signs and symptoms, trauma and injury, etc.

In another exemplary approach, the category scheme may be computed dynamically, which comprises a method for determining the semantic similarity between two concepts. For example, ontologies such as SNOMED and RadLex describe medical knowledge with respect to relationships between concepts. Ontologies describe multiple relationships between concepts used to determine semantic similarity between concepts, and an exemplary type of relationship is the “is-a relationship” in Artificial Intelligence. The “is-a relationship” is a parent-child relationship between concepts; for example, the “left kidney” is-a “kidney,” meaning that the left kidney is a type of kidney. Other exemplary relationships include “has-finding-site” and “is-part-of,” where a “renal cyst” has-finding-site of “kidney,” while a “pons” is-part-of the “brain stem.” That is, the renal cyst may be found at the kidney site, while a pons is a part of the brain stem. In addition, the relationships may be traversed iteratively, where “left kidney” is-a “kidney,” which is the “has-finding-site” relationship reversed. The “renal cyst” and “pons” is-part-of “brainstem,” which in turn is-part-of “brain.” The category scheme derivation engine 113 may be implemented with several engines and modules including, for example, the semantic similarity engine 114 and the dynamic category derivation module 115.

In step 215, the category scheme derivation engine 113 extracts concepts from reports of the imaging exams. In step 216, in an exemplary embodiment, when presented with two concepts from the same ontology, a semantic similarity engine 114, which is part of the category scheme derivation engine 113, indicates the two concepts' semantic similarity. Examples of techniques that may be used to determine semantic similarity may be returning a Boolean answer (yes or no) or generating a numerical value. For example, the semantic similarity engine 114 will return the Boolean “yes” for the pair of concepts “cancer” and “prostate cancer,” indicating that the two concepts are semantically similar, because “cancer” is a generalization of “prostate cancer.” An example of a numerical value may be one-third for the two concepts “cancer” and “prostate cancer” that have three intervening steps in the shortest possible ontology relationship between the two concepts, e.g. “cancer”; X1; X2; “prostate cancer.” Since three steps connect the concepts “cancer” and “prostate cancer,” the inverse of three (one-third) is the numerical value that represents the semantic similarity between the two concepts. As another example, when no ontology relationship connects exemplary concepts A and B, a numerical value representing the semantic similarity between the concepts may be zero. In another exemplary embodiment of step 216, the semantic similarity engine presented with the concept “prostate cancer” will be asked to return all concepts semantically similar to it, where the semantically similar concepts would return the Boolean “yes” or a numerical value exceeding the semantic similarity threshold. In another example, other semantic relationships like “has-finding-site” may be input into the semantic similarity engine to determine the semantic similarity of concepts in the same manner.

In step 217, the dynamic category derivation module 115, which is part of the category scheme derivation engine 113, uses extracted concepts to create groups of similar concepts. In one exemplary embodiment, the dynamic category derivation module 115 assigns a weight to each group of similar concepts, where the weight is proportional to the frequencies of the group's member concepts. In another exemplary embodiment, the dynamic category derivation module 115 assigns a weight to the extracted concept based on the reliability and formality of the data source. For example, concepts extracted from pathology reports have a higher weight than concepts extracted from office notes. In another exemplary embodiment, weights are assigned by the dynamic category derivation module 115 based on the positioning of the term within the ontology, e.g. more general concepts are assigned higher weights. For example, the concept “glioma,” which is a type of cancer tumor, has a lower weight than “cancer,” since “cancer” is more general than “glioma.” A further exemplary embodiment applies a hybrid combination of the above exemplary embodiments in the dynamic category derivation module 115 approach to weight assignment.

Groups with high weights are preferred over groups with low weights. In an exemplary embodiment, a threshold can be established, which sets the maximum number of preferred groups. Groups with high weights may be specialized, e.g. broken down into subgroups, where each subgroup has a lower weight. Groups with low weights may be generalized, e.g. merged with other groups with low weights. Furthermore, each group may have one or more representative concepts, for example, “cancer” and “Non-Hodgkin lymphoma,” and a representative group concept may be the most general concept of the group, e.g. “cancer” instead of “Non-Hodgkin lymphoma.” The specialization and generalization approaches create groups of concepts, so that each group of concepts is a single category scheme.

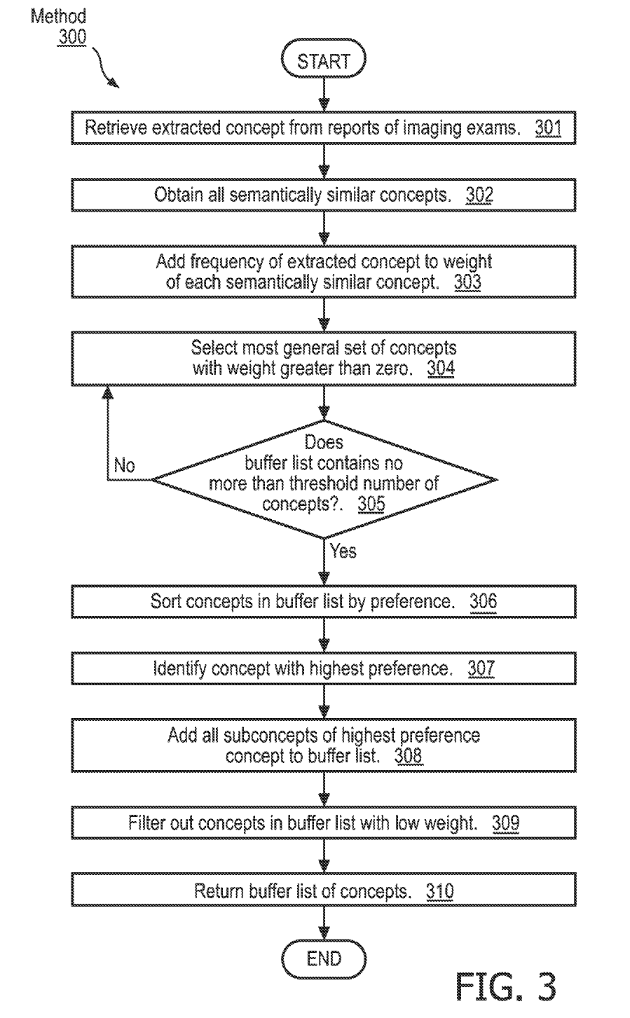

FIG. 3 shows a method for creating concept groups by concept generalization such as in step 217 in FIG. 2 in further detail. In step 301, in an exemplary embodiment, the semantic similarity engine 114 retrieves the extracted concepts from reports of imaging exams. For each of the extracted concepts, in step 302, the semantic similarity engine 114 obtains all concepts semantically similar to the extracted concept. In step 303, the dynamic category derivation module 115 adds the frequency to the weight of each semantically similar concept. For example, the frequency is the number of times the retrieved concept was extracted from the reports of imaging exams. The weight may be the number of semantically similar concepts.

In step 304, the dynamic category derivation module 115 selects the set of concepts with a weight greater than zero, which is the most general concept set, and places this concept set in a buffer list. For example, the most general concept set may be, e.g. the concept set that does not have a more general concept within the “is-a” relationship hierarchy. In step 305, the dynamic category derivation module 115 determines that the buffer list has no more than a threshold number of concepts.

In step 306, the dynamic category derivation module 115 sorts the concepts in the buffer list by preference. For example, a concept with a higher weight is more general, and is a higher preference. In step 307, dynamic category derivation module 115 identifies the concept with the highest preference. In step 308, the dynamic category derivation module 115 adds to the buffer list all subconcepts of the highest preference concept, e.g. all concepts in an “is-a” relationship with the concept of the highest preference.

In step 309, the dynamic category derivation module 115 filters out concepts with lower weight relative to other concepts in the buffer list. In step 310, the dynamic category derivation module 115 returns the buffer list of concepts. Overall, the buffer list of concepts is generalized until no more than a threshold number of concepts remain. The resulting buffer list of concepts is the dynamically derived category scheme.

Returning to FIG. 2, in step 218, the semantic categorization engine 117 assigns one or more semantic categories to an imaging exam, based on its imaging exam report, from the category scheme derived by the category scheme derivation engine 113. A list of ontology concepts is associated with each category. In one exemplary embodiment, the semantic categorization engine 117 matches a given input concept against the category's list of concepts. In another exemplary embodiment, a list of representative concepts is maintained per category, and a semantic categorization subengine attempts to establish a semantic relationship between one input concept and the list of representative concepts through the ontology relationships. Special logic may be applied to restrain the iterative traversal of concepts. For example, a type of logic may stipulate that only the “is-a” relationship may be traversed, or stipulate a particular order of relationship traversal. For example, the logic may require that first, any number of “is-a” relationships may be traversed, then, one “has-finding-site” relationship may be traversed, and next, any number of “is-a” relationships may be traversed. If the ontology may be traversed from the one input concept to one of the category's representative concepts, which respect to the specified traversal logic, the input concept belongs in that category.

In another exemplary embodiment of semantic category assignment, multiple input concepts are categorized together, as a whole. The categories for each individual input concept within the list of input concepts are first obtained, and the outcome is aggregated. Exemplary aggregation methods include placing a list of input concepts in a semantic category if any of the following are true: at least one of the list's input concepts are associated with the category, the majority of the list's input concepts are associated with the category, or all of the list's input concepts are associated with the category. In another exemplary embodiment of categorizing multiple input concepts, the list of category concepts may be externally configurable, so that a user may manipulate concepts that belong to a certain category by modifying the list files. In another exemplary embodiment of categorizing multiple input concepts, the user may add a category by adding a new list of concepts. The semantic categorization engine 114 can then review all concept lists in the input location, and determine semantic category assignments for an imaging exam, based on the list contents.

In step 219, the exam grouping engine 118 groups the current imaging exam with other imaging exams into the same semantic category, based on the output of the semantic categorization engine 117. In one exemplary embodiment, the exam grouping engine 118 determines that two or more imaging exams belong to the same semantic category, if the imaging exams have been associated with the same semantic category through concepts extracted from the imaging exam reports. In another exemplary embodiment, the exam grouping engine 118 groups imaging exams into semantic categories based on contextual parameters including anatomy and modality. In step 219, the exam grouping engine 118 also groups prior stored imaging exams into semantic categories, based on the output of the semantic categorization engine 117, according to the exemplary embodiments described above with reference to grouping the current imaging exam.

In step 220, the relevance reasoning engine 119 identifies prior relevant imaging exams, given a current selected imaging exam. In one exemplary embodiment, the relevance reasoning engine 119 returns all imaging exams that belong to the same semantic category, as determined by the exam grouping engine 118.

In step 221, the user interface (UI) engine 120 displays the timeline of imaging exams, semantic groups and relevant imaging exams, which may be displayed on a display 106.

In step 222, the UI engine 120 aids user navigation of prior relevant imaging exams and other imaging exams on the timeline. The user may navigate the timeline via user interface 104, which may include input devices such as, for example, a keyboard, a mouse, or touch display on the display 106.

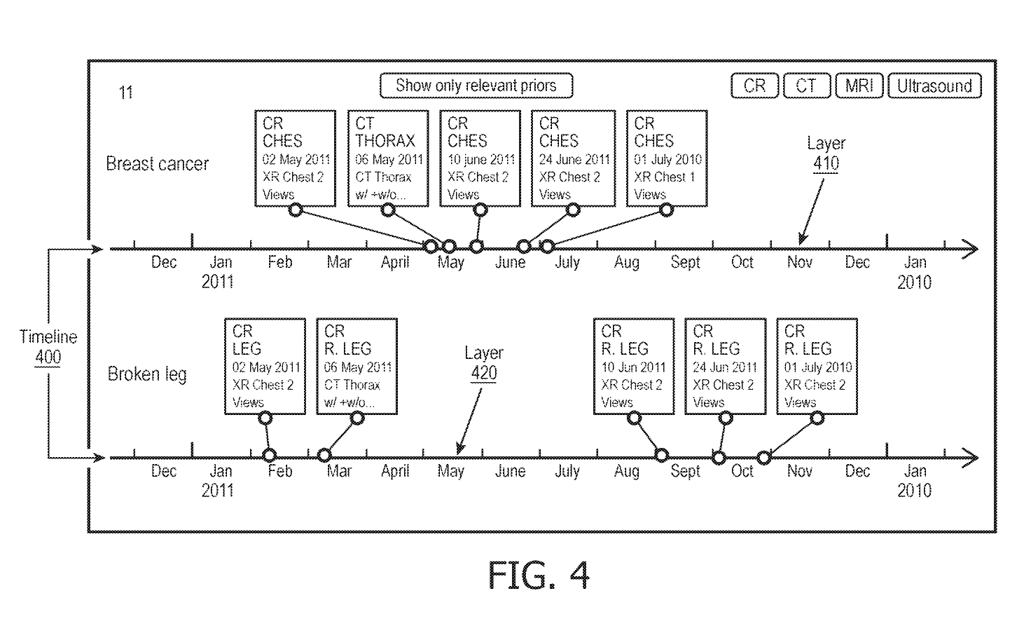

FIG. 4 shows one exemplary embodiment of displaying the timeline on a display 106, where the imaging exam timeline 400 consists of multiple layers, and each layer includes a timeline of the imaging exams belonging to the same semantic group. The imaging exam timeline 400 may include all prior relevant imaging exams, but the separation of the timeline 400 into layer 410 and layer 420 allows the user to review the relevant imaging exams by semantic group. For example, layer 410 includes the imaging exams belonging to the “breast cancer” semantic group, while another layer 420 includes the imaging exams belonging to the “broken leg” semantic group. For example, the exams in layer 410 belonging to the “breast cancer” semantic group may include a computed radiography (CR) scan of the chest in May 2011, a CAT (CT) scan of the thorax in May 2011, another two CR chest scans in June 2011, and a CR chest scan in July 2010. Here, for example, in layer 410, a user may review relevant imaging exams belonging to the “breast cancer” semantic group, including exams of CR chest scans and CT thorax scans, etc. The exams belonging to the “broken leg” semantic group may include, for example, a CR scan of the leg in May 2011, a CR scan of the right leg in May 2011, two CR right leg scans in June 2011, and a CR right leg scan in July 2010. In layer 420, for example, a user may review relevant imaging exams belonging to the “broken leg” semantic group, including exams of CR leg scans, etc.

Thus, from this example, it can be seen that a user interested in viewing the imaging exams related to breast cancer does not have to wade through irrelevant imaging exams (e.g. exams related to a broken leg), and has the relevant imaging exams laid out on a convenient timeline. In addition, since the timeline is not cluttered with irrelevant exams, there is more space available to display details for the relevant imaging exams. Those skilled in the art will understand that the details shown in this figure are only exemplary and the specific details that are shown for the relevant imaging exams may be configurable by the user or the system administrator.

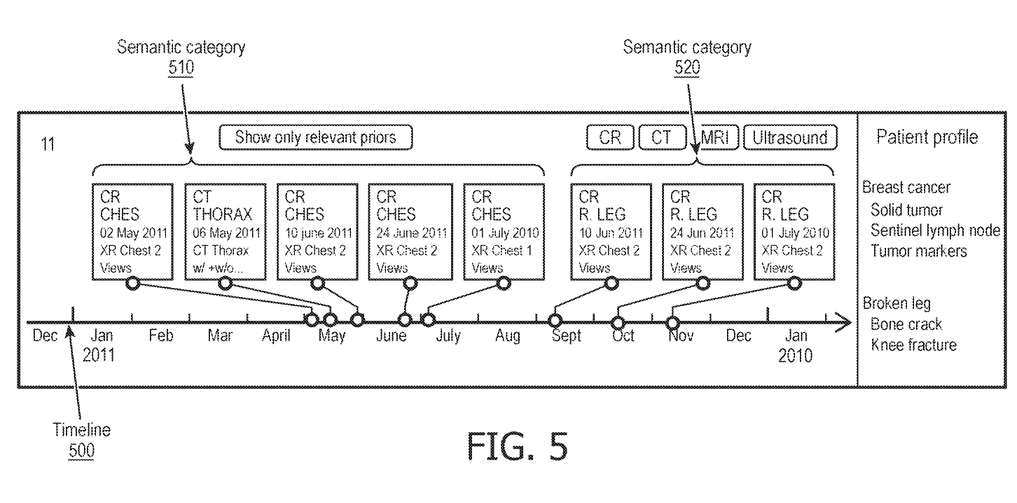

FIG. 5 shows another exemplary embodiment of displaying the timeline on a display 106, which displays the exemplary semantic categories in the vicinity of the imaging exam timeline 500. The imaging exam timeline 500 may include all prior relevant imaging exams, but the visual grouping of the timeline 500 into semantic category 510 and semantic category 520 allows the user to review the relevant imaging exams by semantic group.

For example, in FIG. 5, the exemplary semantic category 510 of “breast cancer” and the exemplary semantic category 520 of “broken leg,” along with the exemplary extracted concepts of solid tumor, sentinel lymph node, and tumor markers for “breast cancer” and bone crack, knee fracture for “broken leg,” are displayed in the vicinity of the imaging exam timeline 500. The display of the exemplary semantic categories (510, 520) in the vicinity of timeline 500 allows the user to review the semantic categories separately, where the semantic categories are grouped with their exemplary respective extracted concepts.

For example, the exams for the exemplary semantic category 510 of “breast cancer” on the timeline 500 may include: a CR chest scan in May 2011, a thorax CT scan in May 2011, two CR chest scans in June 2011, a CR chest scan in July 2010. Here, the visual grouping of exams for semantic category 510 allows the user to review the exams for category 510 of “breast cancer” separately from the other relevant exams on timeline 500. The exams for the exemplary semantic category 520 of “broken leg” on timeline 500 may include two CR right leg scans in June 2011, and a CR right leg scan in July 2010. The visual grouping of exams for semantic category 520 allows the user to review the exams for category 520 of “broken leg” separately from the other relevant exams on timeline 500.

In another exemplary embodiment of this display, the exemplary semantic categories (“breast cancer” (510) and “broken leg” (520)) may be clicked via user interface 104, which highlights pertinent imaging exams or filters out non-pertinent imaging exams on the timeline 500. This highlighting of each semantic category allows the user to review only exams for the semantic category of interest, by visually distinguishing exams for a particular semantic category from the other exams on the timeline 500. The filtering out of non-pertinent imaging exams allows the user to review only pertinent exams for a semantic category of interest, which also visually separates relevant exams for the pertinent semantic category from the other exams on the timeline 500.

In another exemplary embodiment of displaying the timeline on a display 106, the user may click an imaging exam on the timeline, and retrieve all related imaging exams on the timeline, through a user interface 104 control, e.g. a right mouse click to select “show relevant” option within a dropdown menu on the user interface.

In another exemplary embodiment of displaying the timeline on a display 106, semantic reasoning for the categorization process may appear on the timeline. For example, pop-up screens may show the concepts from which the semantic categories were derived. In another exemplary embodiment, the extracted concept may be depicted in the medical narrative context of the report for the imaging exam. In a further exemplary embodiment, the concept or report text may be clicked via user interface 104, which brings the user to the original data source, e.g. pathology reports or office notes.

In a further exemplary embodiment of displaying the timeline on a display 106, selected imaging exams may be expanded on the timeline, where the expanded exams belong to the same semantic category. For example, the user can choose to expand imaging exams for a particular semantic category of interest. As an example, the user can choose to expand the imaging exams on the timeline that belong to the semantic category of “breast cancer.”

Those skilled in the art will understand that the above-described exemplary embodiments may be implemented in any number of manners, including, as a separate software module, as a combination of hardware and software, etc. For example, the report acquisition engine 110, a document parser engine 111, a concept extraction engine 112, a category scheme derivation engine 113, a semantic similarity engine 114 and a dynamic category derivation module 115, a semantic categorization engine 117, an exam grouping engine 118, a relevance reasoning engine 119, and a user interface (UI) engine 120 may be programs containing lines of code that, when compiled, may be executed on a processor.

It will be apparent to those skilled in the art that various modifications may be made to the disclosed exemplary embodiments and methods and alternatives without departing from the spirit or scope of the disclosure. Thus, it is intended that the present disclosure cover the modifications and variations provided that they come within the scope of the appended claims and their equivalents.

1. A method, comprising:
retrieving a report for an imaging exam;
parsing out text from the report;
mapping the parsed text to an ontology;
automatically deriving a categorization scheme from ontology concepts extracted from the report for the imaging exam;
assigning a semantic category to the imaging exam using the ontology concepts and the categorization scheme;
grouping the imaging exam with other imaging exams based on the assigned semantic
determining other imaging exams relevant to the imaging exam, wherein determining includes identifying imaging exams from the same semantic category as the imaging exam; and
displaying on an imaging timeline, the imaging exam and the relevant other imaging exams.

2. (canceled)

3. The method of claim 1, wherein the text includes text headers, the method further comprising:
normalizing the parsed text headers with respect to a pre-determined set of text headers.

4. (canceled)

5. The method of claim 1, wherein automatically deriving the categorization scheme further comprises:
statically determining the categorization scheme by placing imaging exams into predefined categories of the categorization scheme.

6. The method of claim 1, wherein automatically deriving the categorization scheme further comprises:
dynamically computing the categorization scheme, wherein the dynamically computing comprises returning semantically similar concepts and creating groups of similar concepts.

7. The method of claim 6, wherein the returning semantically similar concepts comprises:
in response to an input concept, providing concepts that return a Boolean response “yes” or a numerical value exceeding a threshold.

8. The method of claim 6, wherein the creating groups of similar concepts comprises:
assigning a weight to each group, wherein the weight is proportional to frequencies of member concepts of the group.

9. The method of claim 6, wherein the creating groups of similar concepts comprises:
assigning a weight to a concept based on a reliability of a data source of the concept.

10. The method of claim 6, wherein the creating groups of similar concepts comprises:
assigning a weight to a concept based on a degree of specificity of a concept in the ontology.

11. The method of claim 6, wherein the creating groups of similar concepts comprises a combination of:
assigning a weight to each group, wherein the weight is proportional to frequencies of member concepts of the group;
assigning the weight to the concept based on a reliability of a data source of the concept; and
assigning the weight to the concept based on a degree of specificity of the concept in the ontology.

12. The method of claim 1, wherein the assigning the semantic category to the imaging exam comprises:
associating a list of ontology concepts with each semantic category; and
matching a concept against the list of ontology concepts of the semantic category.

13. The method of claim 1, wherein the assigning the semantic category to the imaging exam further comprises:
maintaining a list of representative ontology concepts for each semantic category;
applying a logic rule to restrain an iterative traversal of concepts; and
determining that an input concept belongs to the semantic category if the input concept traverses the ontology according to the logic rule, from the input concept to one of the representative concepts for the semantic category.

14. The method of claim 1, wherein the assigning the semantic category to the imaging exam comprises:
determining the semantic category for a concept;
aggregating the concepts, wherein the aggregating comprises determining that a list of concepts belong to the semantic category, in at least one of the following situations:
at least one of the concepts on the list are associated with the semantic category;
a majority of the concepts on the list are associated with the semantic category; and
all of the concepts on the list are associated with the semantic category.

15. A system, comprising:
a non-transitory computer readable storage medium storing an executable program; and
a processor executing the executable program to cause the processor to:
retrieve a report for an imaging exam,
parse out text from the report;
map the parsed text to an ontology;
automatically derive a categorization scheme from ontology concepts extracted from the report for the imaging exam further comprising, dynamically computing the categorization scheme, wherein the dynamically computing comprises returning semantically similar concepts and creating groups of similar concepts;
assign a semantic category to the imaging exam using the ontology concepts and the categorization scheme;
group the imaging exams with other imaging exams based on the assigned semantic category
determining other imaging exams relevant to the imaging exam; and
display on an imaging timeline, the imaging exam and the relevant other imaging exams.

16. (canceled)

17. The system of claim 16, wherein the processor executes the executable program to cause the processor to:
determine imaging exams from a same semantic category as relevant to the imaging exam; and
display the imaging timeline with multiple layers, each layer displaying the imaging exams that belong to the same semantic group.

18. (canceled)

19. The system of claim 18, wherein the creating groups of similar concepts comprises one or more of:
assigning a weight to each group, wherein the weight is proportional to frequencies of member concepts of the group;
assigning the weight to the concept based on a reliability of a data source; and
assigning the weight to the concept based on a degree of specificity of the concept in the ontology.

20. (canceled)
